(19) United States
(12) Patent Application Publication
(10) Pub. No.: US 2021/0186715 A1
(43) Pub. Date: Jun. 24, 2021

(54) Prosthetic Foot Cover System

(71) Applicant: Fillauer Composites LLC, Salt Lake City, UT (US)

(72) Inventors: Eric W. Rubie, Salt Lake City, UT (US); Jonathan A. Winegar, Bountiful, UT (US); Phillip M. Thompson, Salt Lake City, UT (US)

(73) Assignee: Fillauer Composites LLC

(21) Appl. No.: 17/124,449

(22) Filed: Dec. 16, 2020

Related U.S. Application Data
(60) Provisional application No. 62/948,621, filed on Dec. 16, 2019.
(60) Provisional application No. 62/948,611, filed on Dec. 16, 2019.

(51) Int. Cl. A61F 2/66; A43B 3/00

(52) U.S. Cl. CPC A61F 2/66; A61F 2002/5001; A61F 2002/6664; A43B 3/0036

(57) ABSTRACT

A prosthetic foot cover device is described. The prosthetic foot cover may include a lattice framework formed to resemble a human foot. The lattice framework has a plurality of lattice cells to form the lattice framework. The prosthetic foot cover may have an inner cavity formed in the lattice framework which is sized and shaped to contain a prosthetic foot, and the inner cavity has an opening to receive the prosthetic foot. The prosthetic foot may have a foot pad that is an outer membrane layer and is joined with a portion of the lattice framework to support a portion of the lattice foot framework.

### PRIORITY DATA

This application claims benefit of U.S. Provisional Patent Application Ser. No. 62/948,611, filed Dec. 16, 2019 and U.S. Provisional Patent Application Ser. No. 62/948,621, filed Dec. 16, 2019, both of which are incorporated herein by reference.

### BACKGROUND

Prostheses (or prosthetics) are artificial devices that replace body parts (e.g., fingers, hands, arms, legs). Generally, prostheses may be used to replace body parts lost by injury or missing from birth. The quality of prostheses has greatly improved in recent years. For example, a prosthetic limb may be molded to have the same shape and density as the person's remaining limb. In addition, elastomeric polymer skins may be used to form the prosthetic limb and give the prosthetic limb a life-like appearance. As another example, improvements in prosthetic limbs may allow for increased feedback and movement.

However, prosthetic limbs still present numerous challenges, particularly in the area of looking and performing as the actual human limbs being replaced. An intact human foot, connected to its ankle, travels through stance and swing phases of a gait cycle during each stride of motion, whether the motion involves walking, jogging, or running. In order to provide higher performance prosthetics, prosthetic feet made of composite materials are often used to provide the energy return characteristics desired by an amputee. For example, a leaf spring composite prosthetic foot may provide desirable characteristics for a foot amputee.

In order to improve the appearance of prosthetic feet, the prosthetic feet are often covered with a cosmetic foot shell that forms an open cavity around the functional prosthetic foot structure. Existing foot shells may be thin-wall hollow shells that do not enhance the prosthetic foot function and often diminish prosthetic foot response. For example, these existing foot shells may be made of polyurethane, PVC (polyvinyl chloride), silicone or other plastics and rubber substitutes. One common problem with foot shells is the internal cavity of the foot shell does not securely hold the prosthetic foot, composite leaf spring foot or other prosthetic foot elements in place which causes the foot to be loosely connected to the foot shell and amputee's shoe. This lack of a secure connection may impede feedback to the user and decrease overall performance of the prosthetic foot.

### BRIEF DESCRIPTION OF THE DRAWINGS

FIG. 1 illustrates an example of a prosthetic foot cover device for a prosthetic foot.

FIG. 2A illustrates an example of one half of a lattice foot framework that is separate from an outer membrane layer.

FIG. 2B illustrates an example of a side view of a full prosthetic foot cover.

FIG. 2C illustrates an example of an isometric view of a full prosthetic foot cover.

FIG. 3 illustrates a prosthetic foot to show the relation between the prosthetic foot, the lattice foot framework and the outer membrane layer.

FIG. 4 illustrates an example of an outer membrane layer joined with the lattice foot framework.

FIG. 5 illustrates an example of lattice sub-shapes and sub-areas which may be formed into a lattice structure of separate and non-uniform density.

FIG. 6 illustrates an example of an outer membrane layer and lattice core formed in single piece or solid shell.

FIG. 7 illustrates an example a prosthetic foot cover device where the lattice framework conforms to the outer membrane layer with a defined thickness.

FIG. 8 illustrates a configuration similar to FIG. 7 where the lattice framework is more sparse or less dense as compared to the density of the lattice framework in FIG. 7.

FIG. 9 illustrates the lattice foot framework where the lattice sub-structures are a plurality of curved walls attached to the outer membrane layer.

FIG. 10 illustrates a side view of a foot framework where the sub-structures are an open cavity design with an internal support structure.

FIG. 11 illustrates an example of a lattice structure that is an efficient lattice structure as measured by the foot cover's strength to weight ratio.

FIG. 12 is a perspective view of a prosthetic foot cover illustrating the shape of the walls of the lattice foot framework at the outside edges of the foot cover.

FIG. 13 is a cross-sectional side view of an example foot cover.

FIG. 14 illustrates an example of the foot cover where walls and shapes of the lattice structure are oriented in a reverse direction as compared to FIG. 13.

FIG. 15 is a close-up view of a cross-section of a foot cover illustrating a heel pad and arch support in an example of the technology.

FIG. 16 is a close-up view of a cross-section of the foot cover illustrating an example of a ball pad and toe pad.

FIG. 17 illustrates an example of a heel channel or heel split lock in a center of the heel of an inner cavity of a foot cover.

FIG. 18 illustrates two heel splits and a forefront split in a foot cover.

FIG. 19 is a perspective view of multiple lattice cells of a lattice framework in an example of the technology.

FIG. 20A is a side view of an example of multiple cells for an open lattice framework.

FIG. 20B is a top view of an example of multiple diamond shaped cells for an open lattice framework.

FIG. 21 is a cross-sectional view of diamond shaped cells in an open lattice framework of a foot cover.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure are to be considered within the scope of the description.

The present technology provides a prosthetic foot cover with an integrated framework, lattice framework(s) or lattice networks. The integrated frameworks and lattices can be made using an additive manufacturing process or a 3D (three-dimensional) printing process. The integrated lattice frameworks may be made of an elastic polymer, another flexible material or a semi-rigid polymer material that can used in a 3D printing process.

Additive manufacturing (AM) may be used to create intricate lattice structures to meet a wide variety of design requirements in a prosthetic limb. AM offers the opportunity to create generative lattice structures for a variety of prosthetic foot designs. Intricate lattice features that were once difficult to produce using injection molding can now be integrated into prosthetic limbs to optimize performance. The benefits of lattice structures can be further used to enable mass customization of prosthetic limbs and more specifically prosthetic feet.

Most prosthetic feet have been covered with a cosmetic foot shell that creates an open cavity around the foot structure. In the past, foot shells have been thin-wall hollow shells that do not enhance the prosthetic foot function and often diminish prosthetic foot response. These thin walls were also solid walls. A common problem with foot shells is that the internal cavity does not securely hold the prosthetic foot elements (e.g., composite leaf spring) of a prosthetic foot in place which may cause the foot to only be loosely connected to the cosmetic foot shell and may impede feedback to the user. Therefore, a secondary foam filler may be used to fix the prosthetic foot in place within the foot shell in order to remedy this issue, but this foam filler tends to inhibit the movement of the prosthetic foot inside the shell.

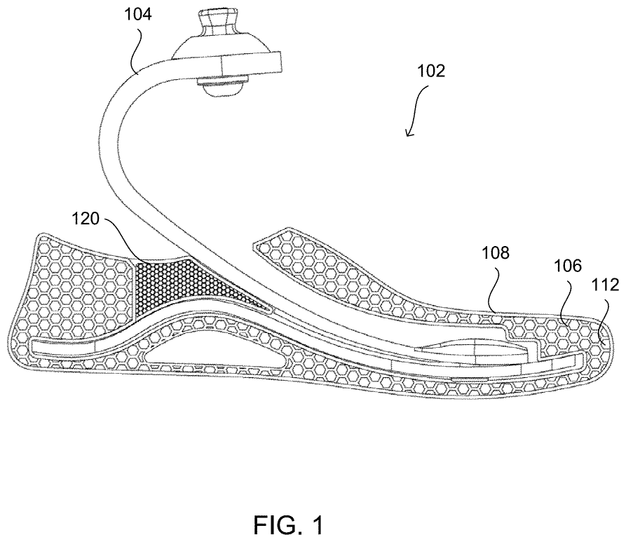

FIG. 1 illustrates a prosthetic foot cover 102 for a prosthetic foot 104. For example, the prosthetic foot may be a composite leaf spring foot. A lattice framework 106 may be provided as a foot cover and the lattice framework may be formed in the three-dimensional shape or cross-section of a human foot or another shape approximating a human foot. The lattice framework may be a lattice foot framework. The lattice cells 112 or lattice sub-elements may be formed in any number of shapes. The hexagonal shape shown in FIG. 1 may be one shape for a lattice foot framework but any other geometric shapes (e.g., rectangles, triangles, circles, diamonds, hexagons, octagons, irregular polygons, other geometric shapes, 3D spine shapes, complex repetitive shapes, organic shapes (e.g., irregular, asymmetric, or curvy), fractal shapes, etc.) may be used. In this configuration, a thin-wall lattice membrane may also be used to replace the solid foam cores of prior foot shells. A membrane design with a lattice core makes producing a lighter and more responsive foot shell feasible. Thus, an amputee may have a prosthetic foot that has the volume of a human foot but the prosthetic foot may still be light weight.

An outer membrane layer 108 may be disposed over the lattice foot framework 106 to cover and support the lattice foot framework 106. The prosthetic foot cover may have a thin-wall lattice membrane to cover the lattice foot framework. The outer membrane layer 108 may also be removable. In another configuration, the outer membrane layer 108 may be fixed to or additively formed with the lattice foot framework 106. The lattice structure of the lattice framework 106 may be a uniform lattice where the lattice sub-shapes are uniform in size. Alternatively, the lattice foot framework 106 may include lattices of varying sizes 120, as will be discussed in more detail later.

In the past, passive prosthetic foot systems have had limited potential to adjust the functional characteristics of the prosthetic system since the systems have often relied on elastomeric inserts in a foot cover to achieve a desired response. In the present technology, the foot cover can be used to adjust the prosthetic foot and may be utilized as a functional member of the foot system to provide enhanced performance to an amputee. The foot cover can also offer a means to regulate and control the foot system by controlling the density of the lattice that is used. The design of lattice foot structure may be tailored to provide a specific compliance (or stiffness) so that the entire gait cycle is optimized to the user's needs.

FIG. 1 also illustrates that heel dampeners and/or bumpers 120 can utilize a lattice structure design that is integrated into the foot cover. The use of a heel dampener or bumper 120 as part of the prosthetic foot cover 102 may be used to change the operating characteristics of the overall prosthetic foot. For example, the heel dampener may change the operating characteristics of a composite leaf spring foot.

In one configuration of the technology, the foot cover may be additively manufactured by selective laser sintering (SLS) which fuses a polymer powder or plastic powder (e.g., nylon or polyamide) laid down in powder layers into a foot cover structure using a computer guided laser that activates layers of the powder. This configuration of the present technology allows the lattice foot framework to be created using SLS but since the outer membrane layer is separate, this allows the outer membrane layer to be removed and the polymer powder can be easily emptied from the lattice. Otherwise, if the outer membrane layer and lattice are manufactured together using SLS, the powder and its added weight may be difficult to remove from the foot cover. The outer membrane layer can be removed after the outer membrane is manufactured in the same powder volume or powder stack, or the outer membrane layer can be manufactured separately so that the powder can be removed from lattice foot framework.

Alternatively, the outer membrane layer can be exchanged for any outer membrane layer that is made by any manufacturing process (e.g., additive, injection, or otherwise). This may provide any cosmetic appearance that an amputee desires. For example, a foot cover can be provided that is larger than the lattice foot framework. Then the foot cover can be placed over the lattice foot framework and the foot cover can be heated (e.g., boiled in water or using heated air) to shrink the foot cover to fit over the lattice foot framework.

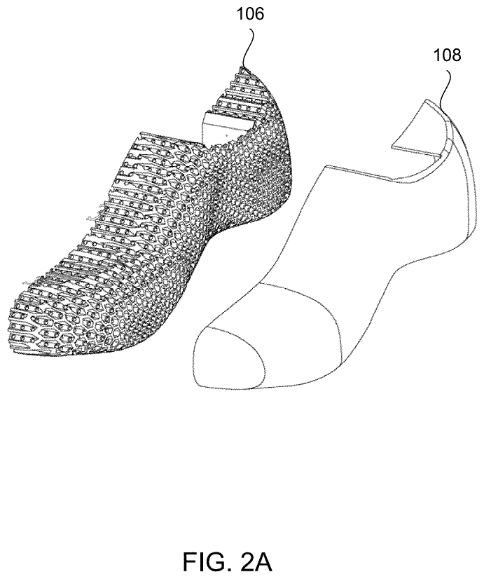

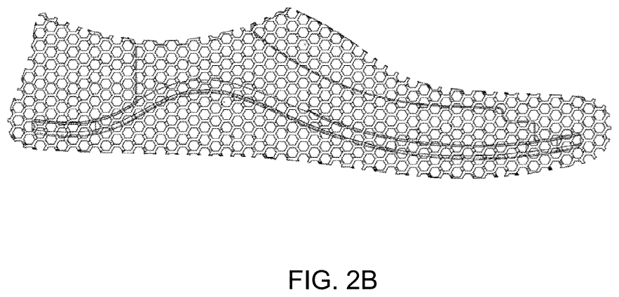

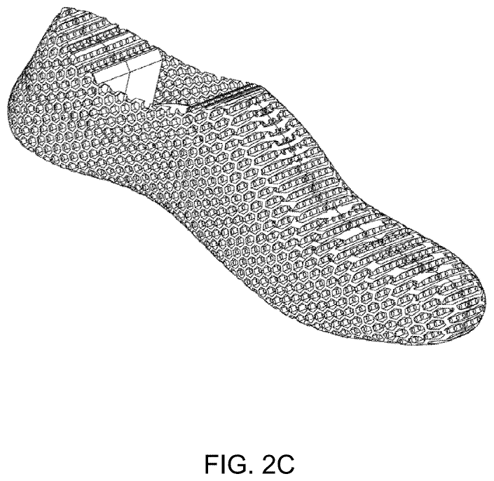

FIG. 2A illustrates that a lattice foot framework 106 may be removed and/or separated from the outer membrane layer 108. This may allow for the lattice foot frameworks 106 of differing densities to be inserted into the outer membrane layer. If an amputee needs a greater density in the lattice foot framework 106 for performing a specific activity, such as running, a lattice foot framework 106 with a greater density (e.g., smaller sub-lattice structure size) may be inserted. If an amputee wants a lattice foot framework 106 for walking, then a lattice foot framework 106 may be inserted that has less overall density. Similarly, the density of the lattice foot framework may be modified depending on the size or weight of the amputee. An amputee that is of greater mass may need a lattice foot framework that is denser and the amputee may be willing to carry the extra weight for the extra support. An amputee that has less mass, such as a youth, may want less support from a lattice foot framework and also want the lower weight that comes with the lower density lattice foot framework.

FIG. 2B illustrates an example of a side view of a full prosthetic foot cover without a membrane cover. FIG. 2C illustrates an example of an isometric view of a full prosthetic foot cover. The foot cover may be a single unitary piece or two halves joined together.

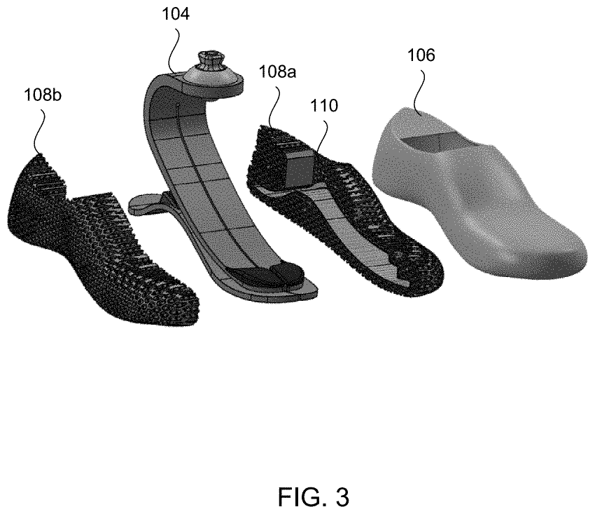

FIG. 3 further illustrates that a prosthetic foot 104 may be inserted into a lattice foot framework that has two halves 108a, 108b or into a lattice foot framework that is a single piece as in FIG. 2C. An inner cavity 110 may be formed in the lattice structure which is sized and shaped to contain or receive the prosthetic foot 104. For example, the inner cavity 110 may have an opening that is sized to receive a composite leaf spring foot. More specifically, the prosthetic foot 104 may be pressed into the inner cavity 110 in a lateral or sideways direction. The lattice foot framework 106 may be provided in two halves so that the prosthetic foot can be slipped into the lattice foot framework 106 easily. Alternatively, the lattice foot framework may be a single piece covering the composite leaf spring foot 104.

The lattice foot framework 108a, 108b and the prosthetic foot 104 may be inserted into or covered by the outer membrane layer 106. The lattice foot framework 108a, 108b and the outer membrane layer 106 may be a single unit, as illustrated, in order to cover the prosthetic foot 104. For example, the lattice foot framework 108a, 108b and the outer membrane layer 106 may work with the prosthetic foot 104 as a system to tune the total response of the prosthetic foot.

FIG. 3 illustrates that the outer membrane layer 108 may have a first half or left half of the outer membrane layer 108. In addition, a right half (not shown) may be provided to cover a right lattice foot framework. In an alternative configuration, the lattice foot framework 106 may be provided in two halves but the outer membrane layer may be a single piece that covers both halves of the lattice foot framework.

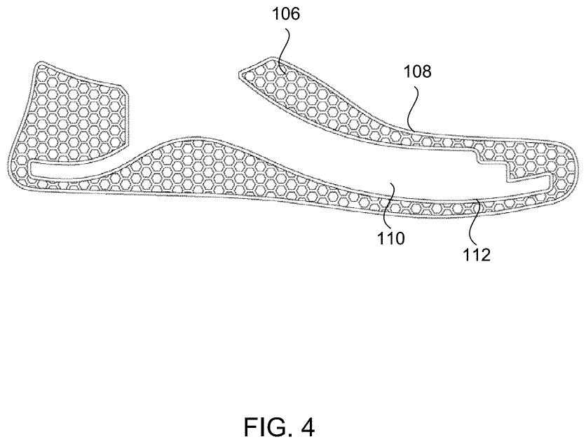

FIG. 4 illustrates a side orthogonal view of the lattice foot framework. The outer membrane 108 may be formed as part of the lattice foot framework. In addition, the outer membrane 108 may also wrap around to form an inner membrane layer 112 for an inner cavity 110 which provides additional support for the prosthetic foot.

The foot cover may be composed of lattice structures with mechanical properties that can be used to enhance the functional characteristics and responsiveness of the prosthetic foot. For example, the foot cover may have a lattice structure that become an integral part of the prosthetic foot. The lattice structures may enable a specific desired response to be integrated into the foot cover. One configuration of this technology incorporates a uniform lattice structure which provides a predictable response due to the homogeneous density. Another variation uses a combination of different density lattices of various densities to generate a variable dynamic response for optimizing the functional characteristics for the user. For example, a first density of lattice cells may exist in a first area and second density of lattice cells may exist in a second area to provide separate response in each area.

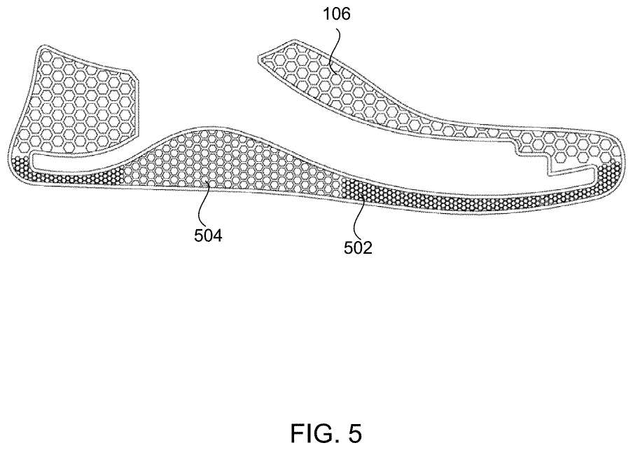

FIG. 5 illustrates that the lattice sub-shapes may be formed into separate areas in a lattice structure so that the overall lattice structure has a non-uniform density or varying density. For example, the lattice structure may have variable densities to generate a variable dynamic response and to optimize functional characteristics for a user. In one configuration, the sub-shapes or cell densities may be smaller in areas 502 of the lattice structure where more support or strength is needed or larger in separate areas 504 where less support is needed.

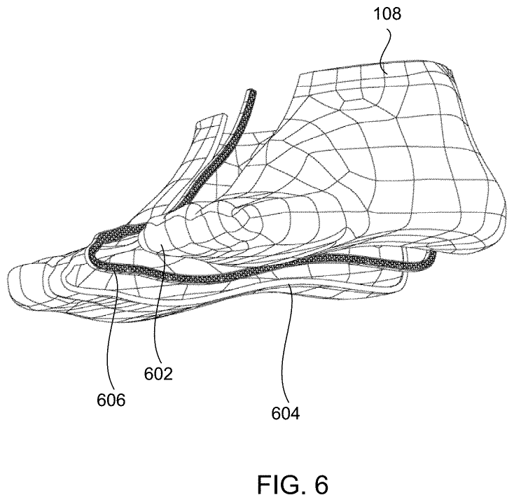

FIG. 6 illustrates a configuration of an outer membrane layer 108 that is designed to appear more anatomically accurate. The outer membrane layer 108 or solid shell may be formed from a single membrane piece and may provide a more anatomically accurate arch 604, set of toes 602, other anatomically accurate aspects of a human foot. FIG. 6 illustrates a cross-sectional portion 606 of the outer membrane layer 108 (for explanatory purposes) that illustrates a lattice network that is attached to the single piece of the more anatomically accurate outer membrane layer 108 or more anatomically accurate foot cover. In one configuration an inner membrane layer may also be included.

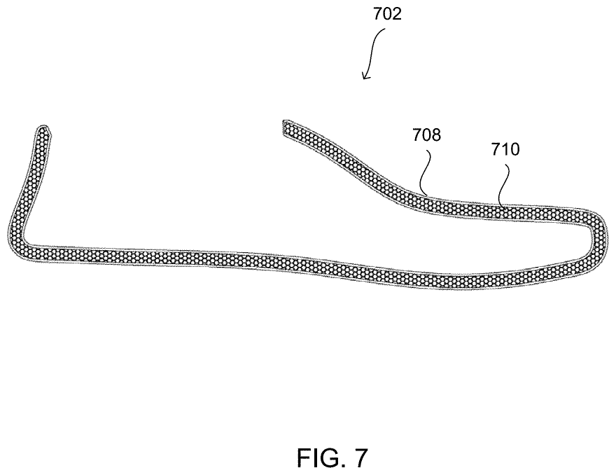

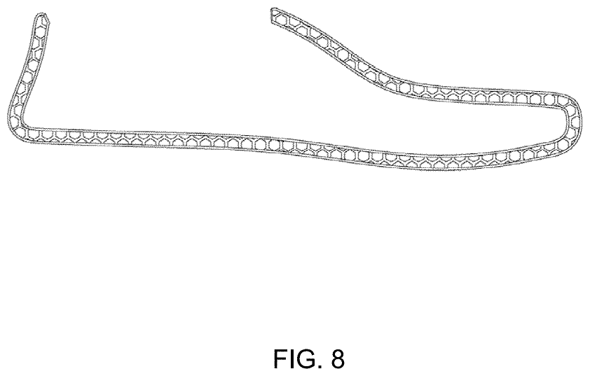

FIG. 7 illustrates a configuration of a prosthetic foot cover device 702 where the lattice framework 710 conforms to the perimeter foot shape that is represented by the outer membrane layer 708. This means the lattice framework 710 is shaped to enclose a volume of a human foot or volume of an anatomical foot shape. In this case, the lattice framework may provide functional support, resilience or cushioning while being just a few millimeters to several centimeters thick, while substantially conforming to the shape of the outer membrane layer 708. FIG. 7 also illustrates that the lattice framework is a relatively dense lattice framework (as compared to earlier figures). FIG. 8 illustrates a configuration similar to FIG. 7 where the lattice framework is relatively sparse or less dense as compared to the density of the lattice framework in FIG. 7.

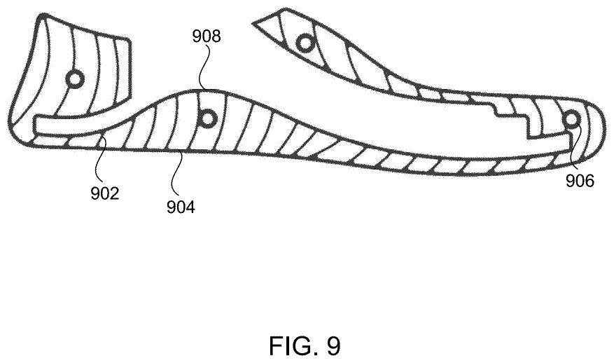

FIG. 9 illustrates the lattice foot framework where the lattice sub-structures are a plurality of arced walls 902 or curved sheets attached to the outer support layer 904 and an inner support layer 908. Alternatively, this lattice foot framework may be supported by an outer support layer 904 that is a mesh and the lattice foot framework may be covered by the outer support layer. For example, the outer support layer 904 may be a mesh of triangular, rectangular, circular, diamond, hexagon, octagon, circle, half moon, irregular polygons, 3D spine shapes, complex repetitive shapes, organic shapes, fractal shapes or other geometric shapes. Connector holes 906 are also illustrated that may be used to connect two halves of the lattice foot framework in FIG. 1 using threaded or press-fit connectors or the foot cover may be a unitary piece.

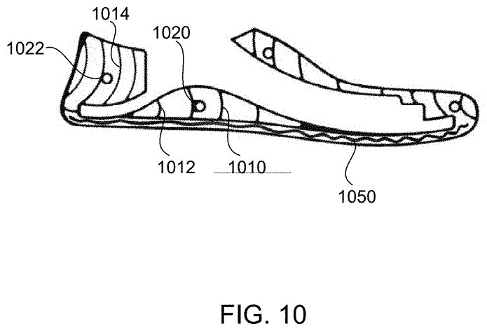

FIG. 10 illustrates a side view of open cavity design with an internal support structure. The open cavity design may include a lattice foot framework where the lattice sub-structures are a plurality of curved support structures 1010, 1012, 1014, curved walls, or curved slabs attached to the outer membrane layer. In addition, the curved supports may be attached to supporting cylinders 1020, 1022 or rods. Other supporting structures such as a curved sheet in a sine wave 1050 may be included to support the ball of the foot.

The open cavity design may be lighter than the lattice foot framework depending on the internal support used. The open cavity design may accommodate a variety of internal structures. The internal structures may be a variety of configurations, such as leaf springs, which can be altered to meet design parameters for a specific amputee. In the case of using resilient leaf springs for the support structure, the resilient leaf springs may offer better cyclic performance (i.e., long-term durability) over time.

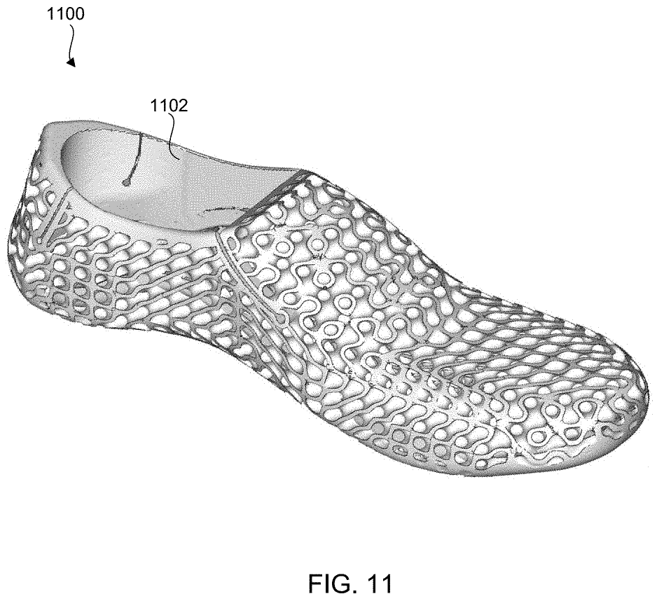

FIG. 11 illustrates a perspective view of a lattice framework 1100 for a foot cover. The foot cover is made from a lattice framework 1100 using lattice cells and may be a single piece foot cover. The lattice foot framework also has a sidewall support layer 1102 which is an inner membrane layer that forms an inner cavity of the lattice foot framework 1100. The sidewall support layer 1102 strengthens the lattice foot framework 1100 to enable the lattice foot framework 1100 to better support the prosthetic foot. The sidewall region of the lattice framework 1100 may be thin in some areas and the sidewall support layer 1102 may add thickness to the sidewall to resist deformation or tearing. The sidewall support layer 102 may also resist fatigue and improve overall durability of the foot cover.

In some configurations as described earlier, the lattice structure may be a variable lattice structure where portions of the lattice structure have a different density of the mesh and wall thickness as compared to other parts of the lattice structure. The lattice foot framework may have a variable material stiffness in separate areas throughout the cover due to the ability to create customizable lattice density and member thickness by using an additive manufacturing process. The material stiffness can be tuned and optimized to maximize desirable performance characteristics such as energy return or high frequency vibration dampening.

The additive manufacturing process enables the cover and the cavity formed by the sidewall support layer 1102 to be custom fit to each prosthetic foot model. This prosthetic foot shape may vary from user to user, activity to activity or from year to year as the prosthetic foot design changes. Being able to conform the cover to the prosthetic foot, enables the foot cover to: better stay on the prosthetic foot, provide a more natural look and provide a more natural feel when walking or running.

The lattice structure illustrated in FIG. 11 depicts a lattice structure that is an efficient lattice structure as measured by the foot cover's strength to weight ratio. In some further configurations, the open lattice structure for the foot cover may have 20% less weight than a comparable solid foot cover. In addition, the lattice illustrated in FIG. 11 is resistant to material fatigue and performs well in a fatigued state.

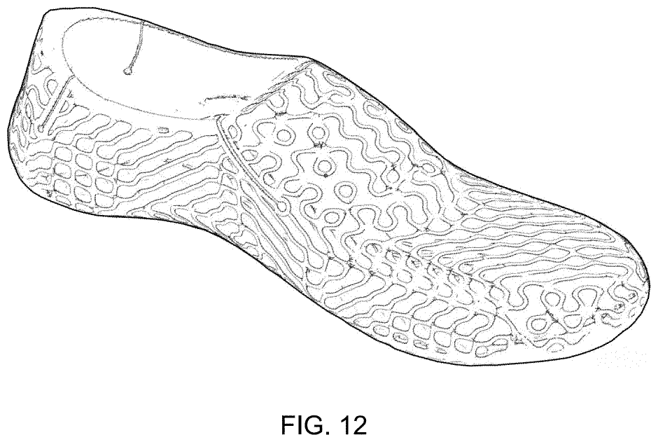

The lattice structure illustrated can match the stiffness of polyurethane foam by tuning lattice density and member thickness. For example, some structure arrangements of the open lattice structure may target the stiffness of the foam to within 1% to 10% of the stiffness of the foam. In this example, the open lattice foot cover may be made of thermoplastic polyamide elastomer that uses polyamide for hard segments and polyether for soft segments that replicate the response of polyurethane. In addition, the lattice structure may be able to approximate the stiffnesses of many linear elastic materials (e.g., natural rubber, synthetic rubber, silicone rubber, aluminum, steel, etc.). FIG. 12 is a perspective view illustrating an example the shape of the lattice walls in the lattice framework at the outside edges of the foot cover.

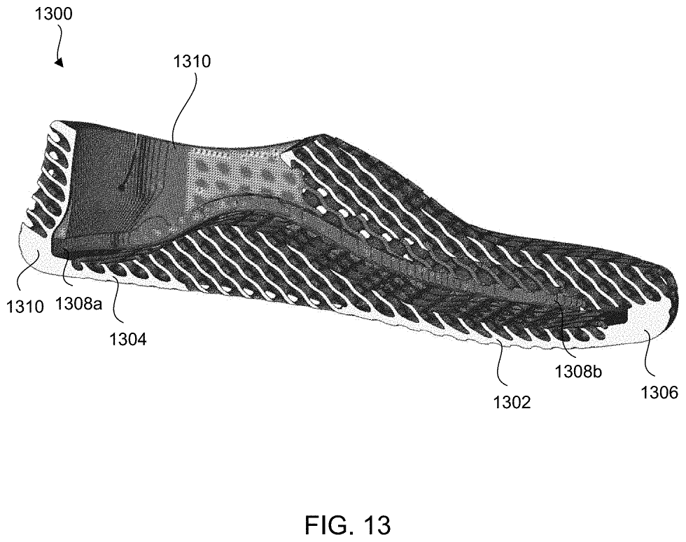

FIG. 13 is a cross-sectional side view of an example of the foot cover 1300. A ball pad 1302 is illustrated that acts as an outer membrane pad for the ball of the foot. The ball pad 1302 as joined with the lattice structure provides the additional strength of the ball pad 1302 along with the reduced weight and strength of the customizable open lattice structure. Similarly, a toe pad or toe block 1306 may be provided that is attached to the lattice structure to protect the lattice from being damaged during use of the foot cover with the prosthetic foot. The heel contact area and toe off area may have additional loads on those points even though the majority of the gait cycle applies loads across the entire lattice. Creating increased strength using the pads and the lattice structure at the heel and toe off points improves the performance of the foot cover. The area of the foot cover that supports the most foot roll over may not need a support pad and may be made with more compliance to provide a softer feel during foot roll over. This compliance is possible in the roll over area because the load is spread out more evenly throughout the lattice structure.

A solid support ring 1308a-b in the inner cavity 1310 can be provided at locations where a foot plate from the prosthetic plate is expected to contact the inner cavity 1310. This solid support ring 1308a-b can increase rigidity and prevent the prosthetic foot from stretching the foot cover out. The solid support ring 1308a-b may also prevent the foot cover from becoming loose.

This foot cover allows variable stiffness along the length or on the height by varying the density of the open lattice structure. Customizable stiffness in certain areas may be provided for an amputee as needed. In addition, the open lattice structure may be customizable for an amputee for their anatomy, by their weight or for specific activities.

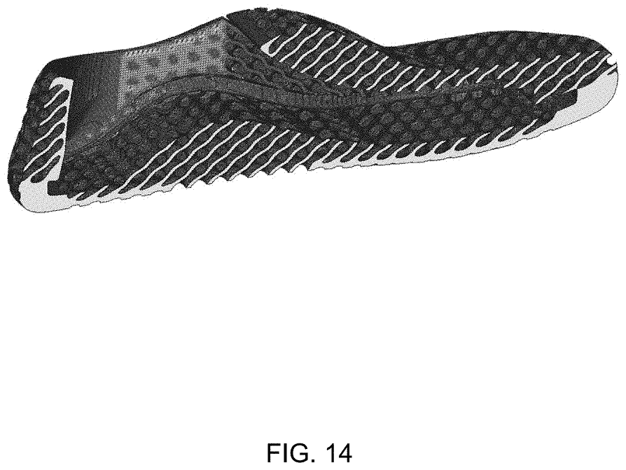

In addition, a heel pad 1304 may be formed at the base of the heel area for the foot cover 1300. The size, depth and dimensions of the heel pad may be varied depending the desired characteristics of the foot cover. A solid heel block 1310 can help avoid the propagation of lattice failure because the heel of the foot may otherwise produce loads that can start to propagate cracks through the lattice structure. In addition, a solid heel block 1310 or internal heel core may stop the propagation of failure at the sharp corner of the heel which can otherwise start to propagate cracks under load. Such solid pieces in strategic locations can increase the fatigue life and durability for the heel pad and overall heel. FIG. 14 illustrates an example of the foot cover where the lattice cell walls and shapes of the lattice structure are oriented in a reversed direction as compared to FIG. 13.

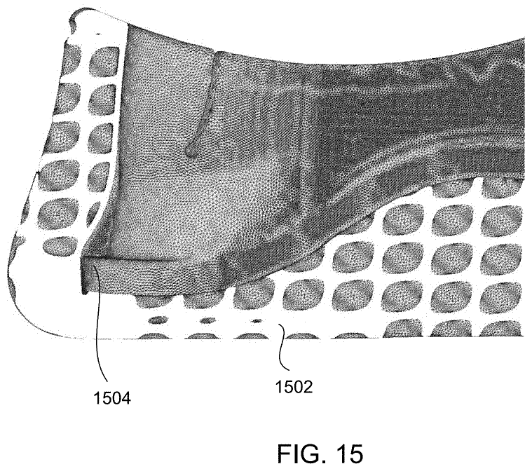

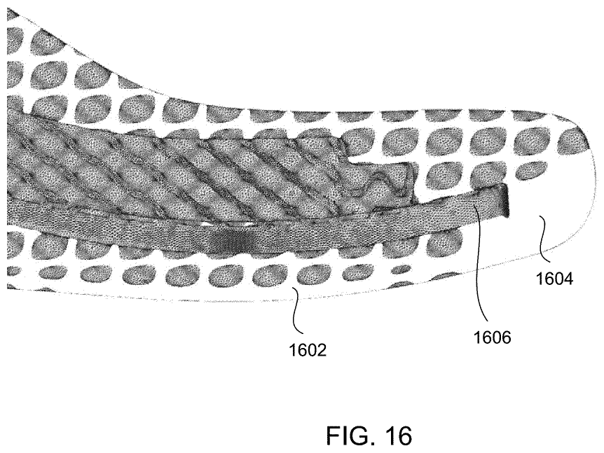

FIG. 15 is a close-up view of a cross-section of the foot cover illustrating an example of a heel pad 1520 and arch support. FIG. 16 is a close-up view of a cross-section of the foot cover illustrating an example of a ball pad 1602 and toe pad 1604 or toe block.

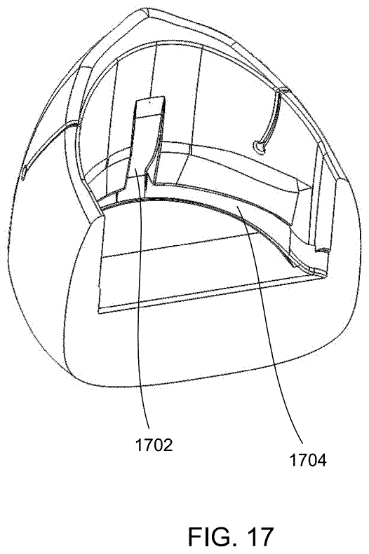

FIG. 17 illustrates an example of a heel channel 1702 or heel split lock in a center of the heel of an inner cavity of a foot cover. Further, a heel groove 1704 is illustrated in the foot cover that is located at or near the heel joint between the heel wall and the bottom wall of the inner cavity of the foot cover. The heel groove 1704 may be formed as an undercut into the heel wall, and the heel groove 1704 may join to the floor of the inner cavity of the foot cover with a radius or curve to the point where actual the joint occurs. This may be considered a rounded internal corner or an internal fillet corner. The heel groove 1704 can reduce cracking and fatigue that may start at the heel joint. FIG. 15 also illustrates a cross-sectional view of the heel groove 1504. Similarly, a toe groove 1606 can be seen in FIG. 16 that operates to provide the same results as the heel groove 1704.

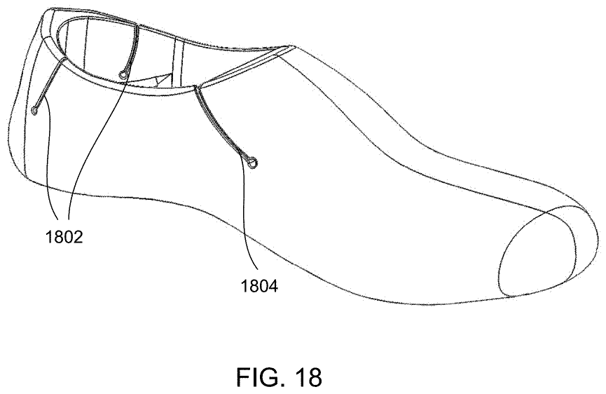

FIG. 18 illustrates two heel splits 1802 and a forefront split 1804 (a symmetric forefront split is hidden) in a foot cover. The solid looking part of the foot cover of FIG. 18 may represent the lattice structure. The addition of forefoot splits 1804 and heel splits 1802 enables easier donning and doffing of the foot cover with respect to a prosthetic foot.

The use of an open lattice design allows for easy evacuation of liquids and foreign objects (e.g., dirt, rocks, sticks, etc.) that may enter the lattice framework. The drainage of the open lattice design can be very useful in a submerged liquid setting such as in boating, at the beach, in the pool, during scuba diving, etc. In the past, other solid foot covers have acted like a bucket by filling with water and the amputee then has to remove the foot cover to drain the foot cover of water. Such problems are avoided with an open lattice framework.

An open lattice design for a foot cover also allows for simple cleaning of the foot cover with water or compressed air because water or air can easily enter the lattice for cleaning and then drain out or leave the open lattice framework. Such cleaning may take place without having to remove the prosthetic foot cover.

In addition, the open lattice structure may be manufactured by selective laser sintering of powder (as described earlier) to make the lattice structure. The open lattice structure makes removing or evacuating the powder from the selective laser sintering process straightforward because the unused powder can more easily escape from the open lattice structure.

The open lattice design also allows the weight of the cover to be optimized while still maintaining critical performance characteristics, such as strength, resilience and durability. Furthermore, when the open lattice structure is manufactured as a single-piece design then any need for additional hardware to affix or hold the foot cover on the prosthetic foot is avoided.

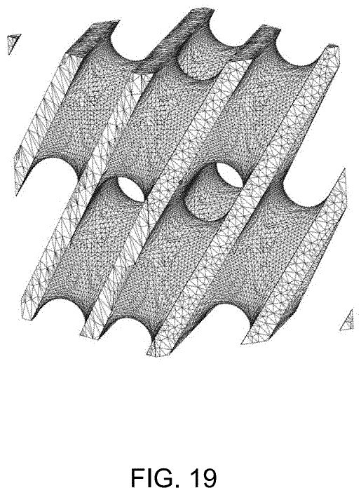

FIG. 19 is a perspective view of an example of multiple lattice cells of an open lattice framework that may be used with the present technology. The lattice framework can be made of structurally defined three-dimensional unit cells that extend in three directions asymmetrically or symmetrically. For example, each cell may be symmetric in three orthogonal directions (e.g., three axes or X, Y and Z dimensions). The open lattice structures as illustrated may be constructed to behave as a foam or a homogenous material even though each of the multiple cells in the lattice contain a defined amount of empty space. By defining the wall thicknesses and the mesh density of the cells, then a stiffness for the lattice framework may be similar to existing foot covers made of solid plastic or rubber. The density of the mesh and wall thickness allows the open lattice framework to be modified in stiffness, compliance, strength and other physical characteristics.

While FIG. 19 illustrates an example of a lattice cell, other types of shapes may be used. An example unit cell may be 18 millimeters by 14 millimeters by 18 millimeters. However, the size of the cells may vary from 13 millimeters in a dimension to 30 millimeters in a dimension (e.g., X, Y or Z). The wall of the cell as illustrated may be 2.5 millimeters, but the wall of a cell may range from 1.0 millimeters to 6.0 millimeters. The lattice cell shapes may be Triply Periodic Minimal Surfaces (TPMS) such Diamond, Gyroid, Neovius, Schwarz, Lidionoid. Furthermore, the lattice cell shapes may be beam and node style lattices such as Simple Cubic, Octet, Kelvin Cell, or Iso-Truss, and other shapes such as a diamond, a box, a pyramid, a hex, a pentagon, a circle, a triangle, or other geometric shapes.

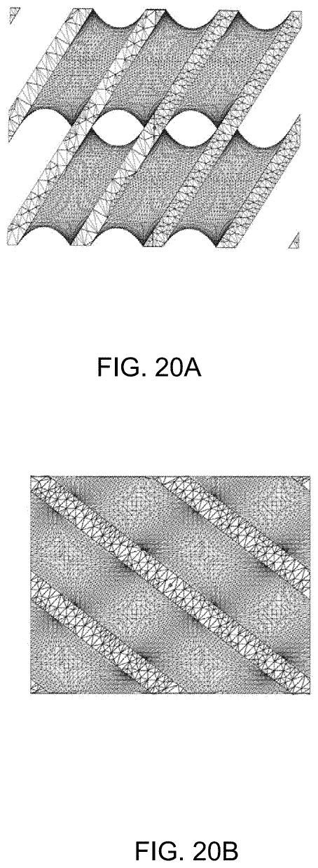

FIG. 20A is a side view of an example of multiple cells for an open lattice framework. FIG. 20B is a top view of multiple diamond shaped cells.

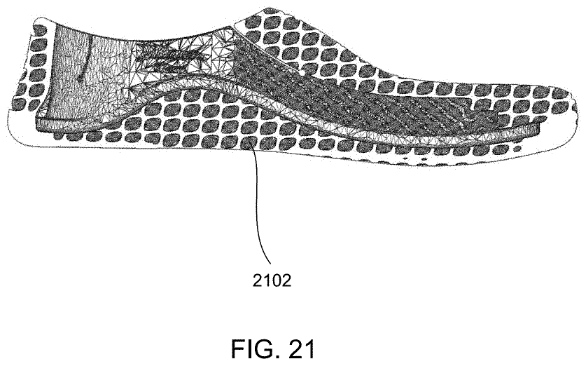

FIG. 21 is a cross-sectional view of diamond shaped cells in an open lattice framework of a foot cover. The mesh lines in FIG. 21 represent a pattern for additively manufacturing the foot cover. Each cell may represent an approximation of a diamond 2102 and many repeating cells in 3 axes can form the open lattice framework.

A lattice framework can also be incorporated in activity specific footwear and soles can be created using a lattice structure, which enables better adaption by amputees. In addition, amputees may no longer be restricted to only wearing existing types of shoes over a foot cover. For example, instead of providing a foot cover that looks like a human foot, a foot cover that looks like a shoe and has a sole with a lattice structure may be manufactured. In another example, the foot cover may have the appearance of a foot and a shoe may be worn over the foot cover but both the lattice framework in the foot cover and the shoe sole may be tuned for the individual amputee and the activities in which the amputee participates. Thus, customized sport soles can be designed for activity specific products to optimize user performance.

Prosthetic users can benefit from mass customization where the clinician may specify the desired compliance of the system for the user based on the results of a clinical evaluation. Therefore, the clinician can digitally select the design features to be incorporated into the prosthetic foot cover to achieve the desired foot response and then use generative design software to optimize the prosthetic foot cover for the user. The clinician can further adjust and fine tune the prosthetic device by creating a customized foot cover for the patient.

The lattice frameworks can also be used for creating mid-soles and outer soles which are designed for specific uses and for the shoes which are used with the prosthetic foot systems. With the advent of mass customization in shoes, the midsole of shoes may be generated and integrate into the overall prosthetic foot system. For example, sport specific soles which may incorporate a lattice structure tuned for participating in sporting events and such soles can be fabricated via AM (additive manufacturing).

The ability to create custom foot lattice structure enables the selective integration of complex lattice structures into a foot cover to regulate and control foot performance. For example, the lattice structure or lattice density can be discretely positioned throughout the prosthetic foot cover to achieve a desired response according to the user's needs and to produce a controlled response.

An Infinitely Variable Lattice Structure (IVLS) also provides the ability to selectively modify and alter the density/geometry of regions of the lattice foot framework to achieve the desired functional characteristics of the prosthetic foot.

In addition, the rollover characteristics of the foot system can be regulated and tuned by strategically positioning the lattice structure throughout the foot cover. For example, the tuning of the prosthetic foot may be considered a Lattice Tuned Design (LTD). Hollow chambers or highly compliant lattice structures can be used on the plantar surface of the cover to function as shock absorbers.

Another aspect of passive prosthetic foot systems is that there is limited potential to adjust the functional characteristics since they often rely on elastomeric inserts to achieve a desired response. Therefore, the present foot cover can be used to adjust the prosthetic foot and can be utilized as a functional member of the foot system which provides enhanced performance. The foot cover can also provide a means to regulate and control the foot system.

The present technology consolidates many individual parts in a single assembly and this in turn may reduce the part count of the prosthetic foot and result in better reliability. For example, a prosthetic foot cover, a lattice, a negative pressure pump and a customizable heel dampener may be contained within one prosthetic foot cover or within a monolithic device created using a single additive manufacturing or printing process.

Even though this technology described above refers to a foot, the same invention can be used in any type of prosthetic or robotic limb flexion device (e.g., any robotic joint). Alternatively, this technology described may be connected to a prosthetic knee system.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A prosthetic foot cover device, comprising:
a lattice framework formed to resemble a human foot, wherein the lattice framework has a plurality of lattice cells to form the lattice framework;
an inner cavity formed in the lattice framework which is sized and shaped to contain a prosthetic foot, wherein the inner cavity has an opening to receive the prosthetic foot; and
a foot pad that is an outer membrane layer and is joined with a portion of the lattice framework to support a portion of the lattice foot framework.

2. The prosthetic foot cover device of claim 1, wherein the foot pad is at least one of: a heel pad, a ball pad, a toe block, or a solid heel block.

3. The prosthetic foot cover device of claim 1, further comprising a solid support ring located in the inner cavity at a location of the prosthetic foot cover where a prosthetic foot contacts the lattice foot framework.

4. The prosthetic foot cover device of claim 1, further comprising a sidewall support layer formed on the inner cavity to support sidewalls of the lattice framework.

5. The prosthetic foot cover device of claim 1, further comprising an outer membrane layer disposed over the lattice framework in at least one area where the prosthetic foot contacts the lattice foot framework to cover and support the lattice foot framework.

6. The prosthetic foot cover device as in claim 1, further comprising lattice cells having a lattice density and a member thickness to simulate polyurethane foam.

7. The prosthetic foot cover device as in claim 1, wherein lattice framework has open lattice cells though which water or compressed air pass.

8. The prosthetic foot cover device as in claim 1, further comprising a heel split lock on an interior of a heel in the inner cavity of the lattice framework.

9. The prosthetic foot cover device as in claim 1, further comprising at least one of a forefoot split or a heel split.

10. A prosthetic foot cover device, comprising:
a lattice foot framework formed in a shape of a human foot, wherein the lattice foot framework has a plurality of lattice cells extending in at least three axes;
an inner cavity formed in the lattice foot framework which is sized and shaped to contain a prosthetic foot, wherein the inner cavity has an opening to receive the prosthetic foot; and
an outer membrane layer disposed over the lattice foot framework to cover and support the lattice foot framework.

11. The prosthetic foot cover as in claim 10, wherein the lattice foot framework is a symmetric lattice or a asymmetric lattice structure.

12. The prosthetic foot cover as in claim 10, wherein the lattice foot framework is a thin-wall lattice membrane.

13. The prosthetic foot cover as in claim 10, wherein the lattice foot framework has a first density of lattice cells in a first area and second density of lattice cells in a second area to generate a variable dynamic response to optimize functional characteristic for a user.

14. The prosthetic foot cover as in claim 10, wherein the lattice foot framework is made of an elastic polymer or a semi-rigid polymer.

15. The prosthetic foot cover as in claim 10, wherein the outer membrane layer is configured to be removable.

16. A prosthetic foot cover device, comprising:
a lattice foot framework formed in a shape of a human foot, wherein the lattice foot framework has a plurality of lattice cells extending in at least three axes to form the lattice network;
a first area in the lattice foot framework of a first cell density;
a second area in the lattice foot framework of a second cell density that is greater than a first area in order to provide increased durability;
an inner cavity formed in the lattice foot framework which is sized and shaped to contain a prosthetic foot, wherein the inner cavity has an opening to receive the prosthetic foot; and
an outer membrane layer disposed over a portion of the lattice foot framework to strengthen the lattice foot framework.

17. The prosthetic foot cover device of claim 16, wherein the outer membrane layer is at least one of: a heel pad, a ball pad or solid heel block.

18. The prosthetic foot cover device of claim 16, further comprising an inner membrane layer disposed over the lattice foot framework in at least one area where the prosthetic foot contacts the lattice foot framework to support the lattice foot framework.

19. The prosthetic foot cover device as in claim 16, further comprising a heel split lock on an interior of a heel of the lattice foot framework.

20. The prosthetic foot cover device as in claim 16, further comprising at least one of a forefoot split or a heel split.
